(19) United States
(12) Patent Application Publication
(10) Pub. No.: US 2024/0374730 A1
(43) Pub. Date: Nov. 14, 2024

(54) PHOTOACTIVATABLE PRODRUG NANOPARTICLES FOR COMBINED ANTI-ANGIOGENESIS AND PHOTODYNAMIC THERAPY

(71) Applicant: The University of Hong Kong, Hong Kong (CN)

(72) Inventors: Weiping WANG, Hong Kong (CN); Kaiqi LONG, Hong Kong (CN); Shuting XU, Hong Kong (CN)

(21) Appl. No.: 18/293,171

(22) PCT Filed: Jul. 25, 2022
(86) PCT No.: PCT/CN2022/107621

§ 371 Date: Jan. 29, 2024

Related U.S. Application Data
(60) Provisional application No. 63/203,614, filed on Jul. 27, 2021.

(51) Int. Cl. A61K 41/00; A61K 9/51; A61K 47/55; A61P 27/02

(52) U.S. Cl. CPC A61K 41/0071; A61K 9/5146; A61K 47/55; A61P 27/02

(57) ABSTRACT

Provided is a singlet oxygen-cleavable anti-VEGF prodrug. Provided is a composition comprising a photosensitizer and biocompatible polymer for the co-assembly of photoactivatable nanoparticles with the singlet oxygen-cleavable anti-VEGF prodrug. This red-light-activatable prodrug composition can be used in methods for the combined anti-angiogenesis and PDT treatment of wet AMD and other diseases, capable of light-controllable drug delivery to the CNV lesions and other disease sites via noninvasive intravenous administration.

### FIELD OF THE INVENTION

This invention is generally in the field of synthetic prodrugs and their use in combined anti-angiogenesis and photodynamic therapy.

### BACKGROUND OF THE INVENTION

Age-related macular degeneration (AMD), a degenerative eye disease that preferentially affects people over age 50, has been regarded as a leading cause of irreversible vision loss worldwide.¹ At least 11 million AMD cases were reported in the United States alone, comparable to the total prevalence of all invasive cancers and resulting in a direct healthcare cost of $4.6 billion per year.² Among them, wet AMD, characterized by progressive choroidal neovascularization (CNV) and retinal damage, contributes to most AMD cases with severe vision impairment.^{3,4} However, due to its complicated pathogenesis, there is no effective method that can achieve a complete cure for wet AMD. The current treatment modalities include anti-vascular endothelial growth factor (anti-VEGF) therapy through intravitreal administration, photodynamic therapy (PDT) with verteporfin, and thermal laser treatment.⁵ Particularly, anti-VEGF therapy has become the first line option for clinical intervention due to the critical role of elevated VEGF levels in pathological CNV growth.⁶ In clinical practice, the anti-VEGF agents are injected directly into the vitreous body via the pars plana, since systemic use of intravenous injection could induce severe adverse events, including arterial hypertension and thromboembolism.^{7,8} Although this treatment modality has been proven to be effective in improving the visual acuity with relatively low risk of systemic adverse events, it still suffers from some shortcomings.⁹ The long-term management of wet AMD commonly requires repeated intravitreal injection of anti-VEGF agents, which not only increases the risks of endophthalmitis, retinal detachment, cataract and other ocular complications caused by intravitreal injection procedure but also increased the mental and financial stress of patients.^{10,11} Therefore, developing noninvasive and targeted delivery techniques is urgently desired for antiangiogenic therapy.

Given that anti-VEGF therapy would become less useful to inhibit mature vasculature, selective vascular occlusion by PDT has been extensively used for the treatment of the later stage of wet AMD.¹² However, thrombosis induced by PDT generally leads to the upregulation of vasoactive factors like VEGF in the CNV lesions, which compromises the effectiveness of PDT.¹³ As a result, the combined therapy of anti-VEGF agents and PDT was introduced and clinical evidence has demonstrated that anti-VEGF and PDT combination therapy could result in improved visual acuity and prolonged stability for wet AMD patients.^{14,15} Thus, effective combinations of anti-angiogenic and anti-vascular therapies are considered to be a more efficient approach for wet AMD treatment, especially for those patients who responded badly to anti-VEGF or PDT treatment alone.^{16, 17}

Therefore, there remains a need for a combination of therapeutics with targeted delivery that exerts effective VEGF inhibition.

### BRIEF SUMMARY OF THE INVENTION

The present invention relates to a light-activatable prodrug composition for use in methods for combined anti-VEGF and PDT treatment of diseases, including, for example, wet AMD, which enables targeted drug delivery to the choroidal neovascularization (CNV) lesions via, for example, noninvasive intravenous injection. In certain embodiments, a singlet oxygen-cleavable dimeric prodrug is provided and can optionally be combined with a photosensitizer, such as, for example, FDA-approved verteporfin and biocompatible polymer, such as, for example, DSPE-PEG₂₀₀₀, for the co-assembly of photoactivatable nanoparticles. In certain embodiments, the prodrug-based nanoassemblies can be used in methods to release an active anti-VEGF drug. In certain embodiments, the prodrug-based nanosystem can be used in methods to treat wet AMD. The methods of the subject invention can also be used for the treatment of other eye diseases, such as, for example, diabetic retinopathy, and solid tumors.

### BRIEF DESCRIPTION OF THE DRAWINGS

The patent or application file contains at least one drawing executed in color. Copies of this patent or patent application publication, with color drawing(s), will be provided by the Office upon request and payment of the necessary fee.

FIG. 1A Schematic illustration of the photoactivatable prodrug-based nanosystem (Di-DAS-VER) for the treatment of wet age-related macular degeneration. FIG. 1B Chemical structure and red light-triggered cleavage of the dimeric prodrug (Di-DAS) involved in the developed nanosystem. Reactive oxygen species (ROS) generated by the excited photosensitizer can lead to the cleavage of thioketal linkage to release anti-VEGF agents. DAS: dasatinib.

FIGS. 2A-2D Characterization of the photoactivatable nanosystem formed by Di-DAS and verteporfin. FIG. 2A DLS analysis of the prodrug-based nanoparticles (Di-DAS-VER). Inserted pictures show the schematic illustration and TEM image of the nanoparticles. Scale bar=200 nm. FIG. 2B Absorption spectra of the dimeric prodrug (Di-DAS), photosensitizer (VER) and nanoparticles (Di-DAS-VER). FIG. 2C Average particle size and polydispersity of Di-DAS-VER in pH 7.4 PBS at 37° C. for 48 h. FIG. 2D Average particle size and polydispersity of Di-DAS-VER in DMEM containing 10% fetal bovine serum (FBS) at 37° C. for 48 h. FIG. 2E Quantitative fluorescence intensity measurement with Singlet Oxygen Sensor Green (SOSG) as singlet oxygen detection probe. Different formulations were exposed to 690 nm laser irradiation (100 mW/cm²) for different periods. FIG. 2F Intracellular ROS generation by free VER and Di-DAS-VER nanoparticles upon illumination. HUVECs were treated with VER-containing formulations (0.1 μM VER) for 4 h, followed by incubation with 20 μM DCFH-DA, and 690 nm irradiation (10 mW/cm², 400 s) or not. Data were presented as mean±SD (n=3). DAS: dasatinib. VER: verteporfin.

FIG. 3 HPLC spectra of Di-DAS-VER nanoparticle solutions (FIG. 3A) with or (FIG. 3B) without 690 nm irradiation at 100 mW/cm² for various periods. Arrows on the left denote DAS; arrows on the right denote Di-DAS. The detection wavelength was 320 nm.

FIGS. 4A-4C In vitro assessment of cell viability and anti-angiogenic activity of Di-DAS and Di-DAS-VER. FIG. 4A Quantitative HUVEC cell viability of dasatinib and Di-DAS by MTT assay. Cells were incubated with 20 ng/mL VEGF₁₆₅ and co-treated with dasatinib or Di-DAS at various concentrations for 48 h. FIG. 4B VEGF-stimulated HUVEC tube formation assay of Di-DAS-VER. HUVEC suspensions were treated with 20 ng/mL VEGF₁₆₅ and co-incubated with different formulations at a DAS equivalent dose of 0.05 μM, followed by 0.75 J/cm² of light irradiation at 690 nm or not. HUVEC tube formation was observed at 4 h after treatment. FIG. 4C Quantitative HUVEC cell viability of Di-DAS-VER by MTT assay. Cells were treated with 20 ng/mL VEGF₁₆₅ and co-incubated with different formulations at a DAS equivalent dose of 1 μM, followed by 4 J/cm² of light irradiation at 690 nm or not. MTT assay was performed at 24 h after treatment. The administrated dose of verteporfin was the same as that of Di-DAS-VER determined by HPLC. Data were presented as mean±SD (n=6). DAS: dasatinib; VER: verteporfin.

FIG. 5 Representative microscopy images (FIG. 5A) and quantitative analysis (FIG. 5B) of HUVEC scratch-wounds at 0 and 20 h after incubation with DAS, Di-DAS, VER and Di-DAS-VER nanoparticles with or without 690 nm laser irradiation. Changes of scratch-wound areas were analyzed by ImageJ to calculate the wound recovery rates. Data were presented as mean±standard deviations (n=5 images/group at each time point).

FIG. 6A Representative images of HUVEC migration assay of Di-DAS at 24 h post-treatment. HUVEC suspensions were seeded in the top chamber and incubated with different formulations at a DAS equivalent dose of 0.05 μM, followed by 0.75 J/cm² of light irradiation at 690 nm or not. FIG. 6B Representative images and the corresponding quantification results of Western blot analysis of Src-related angiogenic signaling proteins in HUVEC after different formulations treatment at an equivalent dose of 1 μM dasatinib at 37° C. for 24 h, with or without 0.75 J/cm² of 690 nm light irradiation. DAS: dasatinib; VER: verteporfin.

FIG. 7 The synthesis route of the dimeric prodrug (Di-DAS).

FIG. 8 ¹H NMR spectrum (FIG. 8A) and mass spectra (FIG. 8B) of Di-DAS.

### DETAILED DISCLOSURE OF THE INVENTION

### Selected Definitions

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The transitional terms/phrases (and any grammatical variations thereof) “comprising”, “comprises”, “comprise”, “consisting essentially of”, “consists essentially of”, “consisting” and “consists” can be used interchangeably.

The phrases “consisting essentially of” or “consists essentially of” indicate that the claim encompasses embodiments containing the specified materials or steps and those that do not materially affect the basic and novel characteristic(s) of the claim.

The term “about” means within an acceptable error range for the particular value as determined by one of ordinary skill in the art, which depends in part on how the value is measured, i.e., the limitations of the measurement system. In the context of compositions containing amounts of ingredients where the terms “about” is used, these compositions contain the stated amount of the ingredient with a variation (error range) of 0-10% around the value (X±10%). In other contexts the term “about” is provides a variation (error range) of 0-10% around a given value (X±10%). As is apparent, this variation represents a range that is up to 10% above or below a given value, for example, X±1%, X±2%, X±3%, X±4%, X±5%, X±6%, X±7%, X±8%, X±9%, or X±10%.

In the present disclosure, ranges are stated in shorthand to avoid having to set out at length and describe each and every value within the range. Any appropriate value within the range can be selected, where appropriate, as the upper value, lower value, or the terminus of the range. For example, a range of 0.1-1.0 represents the terminal values of 0.1 and 1.0, as well as the intermediate values of 0.2, 0.3, 0.4, 0.5, 0.6, 0.7, 0.8, 0.9, and all intermediate ranges encompassed within 0.1-1.0, such as 0.2-0.5, 0.2-0.8, 0.7-1.0, etc. Values having at least two significant digits within a range are envisioned, for example, a range of 5-10 indicates all the values between 5.0 and 10.0 as well as between 5.00 and 10.00 including the terminal values. When ranges are used herein, combinations and subcombinations of ranges (e.g., subranges within the disclosed range) and specific embodiments therein are explicitly included.

As used herein, the term “subject” refers to an animal, needing or desiring delivery of the benefits provided by a therapeutic compound. The animal may be for example, humans, pigs, horses, goats, cats, mice, rats, dogs, apes, fish, chimpanzees, orangutans, guinea pigs, hamsters, cows, sheep, birds, chickens, as well as any other vertebrate or invertebrate. These benefits can include, but are not limited to, the treatment of a health condition, disease or disorder; prevention of a health condition, disease or disorder; immune health; enhancement of the function of an organ, tissue, or system in the body. The preferred subject in the context of this invention is a human. The subject can be of any age or stage of development, including infant, toddler, adolescent, teenager, adult, or senior.

As used herein, the terms “therapeutically-effective amount,” “therapeutically-effective dose,” “effective amount,” and “effective dose” are used to refer to an amount or dose of a compound or composition that, when administered to a subject, is capable of treating or improving a condition, disease, or disorder in a subject or that is capable of providing enhancement in health or function to an organ, tissue, or body system. In other words, when administered to a subject, the amount is “therapeutically effective.” The actual amount will vary depending on a number of factors including, but not limited to, the particular condition, disease, or disorder being treated or improved; the severity of the condition; the particular organ, tissue, or body system of which enhancement in health or function is desired; the weight, height, age, and health of the patient; and the route of administration.

As used herein, the term “treatment” refers to eradicating, reducing, ameliorating, or reversing a sign or symptom of a health condition, disease or disorder to any extent, and includes, but does not require, a complete cure of the condition, disease, or disorder. Treating can be curing, improving, or partially ameliorating a disorder. “Treatment” can also include improving or enhancing a condition or characteristic, for example, bringing the function of a particular system in the body to a heightened state of health or homeostasis.

As used herein, “preventing” a health condition, disease, or disorder refers to avoiding, delaying, forestalling, or minimizing the onset of a particular sign or symptom of the condition, disease, or disorder. Prevention can, but is not required, to be absolute or complete; meaning, the sign or symptom may still develop at a later time. Prevention can include reducing the severity of the onset of such a condition, disease, or disorder, and/or inhibiting the progression of the condition, disease, or disorder to a more severe condition, disease, or disorder.

In some embodiments of the invention, the method comprises administration of multiple doses of the compounds of the subject invention. The method may comprise administration of 1, 2, 3, 4, 5, 6, 7, 8, 9, 10, 15, 20, 25, 30, 35, 40, or more therapeutically effective doses of a composition comprising the compounds of the subject invention as described herein. In some embodiments, doses are administered over the course of 1 day, 2 days, 3 days, 4 days, 5 days, 6 days, 7 days, 10 days, 14 days, 21 days, 30 days, or more than 30 days. The frequency and duration of administration of multiple doses of the compositions is such as to release anti-VEGF drug and PDT treatment or to treat wet AMD, other eye diseases, or tumors. Moreover, treatment of a subject with a therapeutically effective amount of the compounds of the invention can include a single treatment or can include a series of treatments. It will also be appreciated that the effective dosage of a compound used for treatment may increase or decrease over the course of a particular treatment. Changes in dosage may result and become apparent from the results of diagnostic assays or imaging techniques for detecting tumor sizes known in the art. In some embodiments of the invention, the method comprises administration of the compounds at several time per day, including but not limiting to 2 times per day, 3 times per day, and 4 times per day.

As used herein, an “isolated” or “purified” compound is substantially free of other compounds. In certain embodiments, purified compounds are at least 60% by weight (dry weight) of the compound of interest. Preferably, the preparation is at least 75%, more preferably at least 90%, and most preferably at least 99%, by weight of the compound of interest. For example, a purified compound is one that is at least 90%, 91%, 92%, 93%, 94%, 95%, 98%, 99%, or 100% (w/w) of the desired compound by weight. Purity is measured by any appropriate standard method, for example, by column chromatography, thin layer chromatography, or high-performance liquid chromatography (HPLC) analysis.

By “reduces” is meant a negative alteration of at least 1%, 5%, 10%, 25%, 50%, 75%, or 100%.

By “increases” is meant as a positive alteration of at least 1%, 5%, 10%, 25%, 50%, 75%, or 100%.

As used herein, a “pharmaceutical” refers to a compound manufactured for use as a medicinal and/or therapeutic drug.

As used herein, a “prodrug” is a compound that may be converted to a pharmaceutically active compound after administration to a subject.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

Other features and advantages of the invention will be apparent from the following description of the preferred embodiments thereof, and from the claims.

### Preparation of Prodrug and Compositions Thereof

In certain embodiments, a singlet oxygen-cleavable anti-VEGF prodrug can be synthesized. A reactive oxygen species (ROS)-cleavable dimeric prodrug can be synthesized by conjugating two molecules of an anti-VEGF agent or two anti-VEGF agents via a ROS-sensitive linker, such as, for example, a thioketal linker, thioether linker, peroxalate ester linker, aminoacrylate linker, selenide/diselenide or telluride-containing linker. The anti-VEGF agent can be a small-molecule anti-angiogenic inhibitor containing carboxy or hydroxyl groups, such as, for example, dasatinib (DAS), orantinib, SU5402, and/or CGP60474. In certain embodiments, two different anti-VEGF agents can be linked via a ROS-sensitive linker.

The following is the chemical formulas of prodrugs of the subject invention:

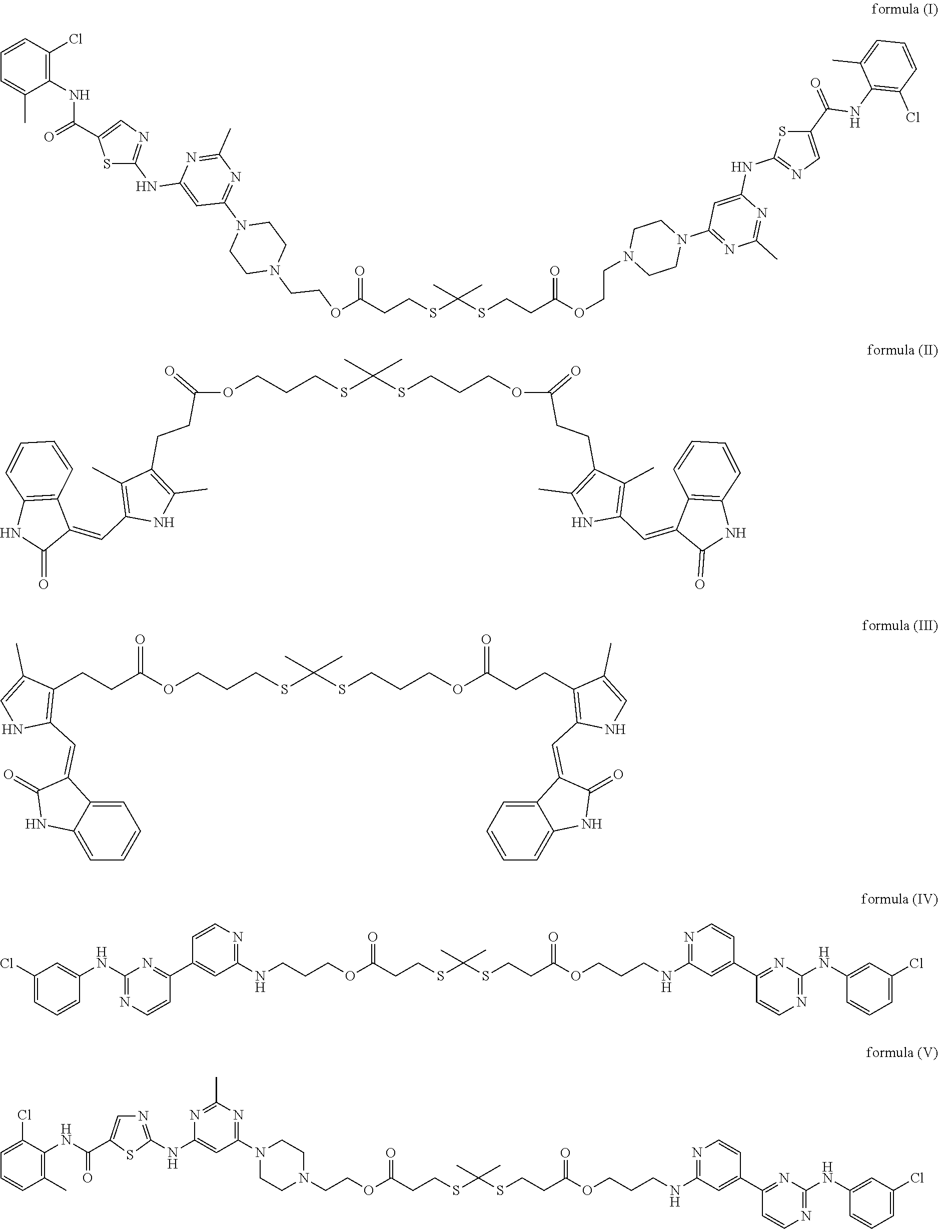

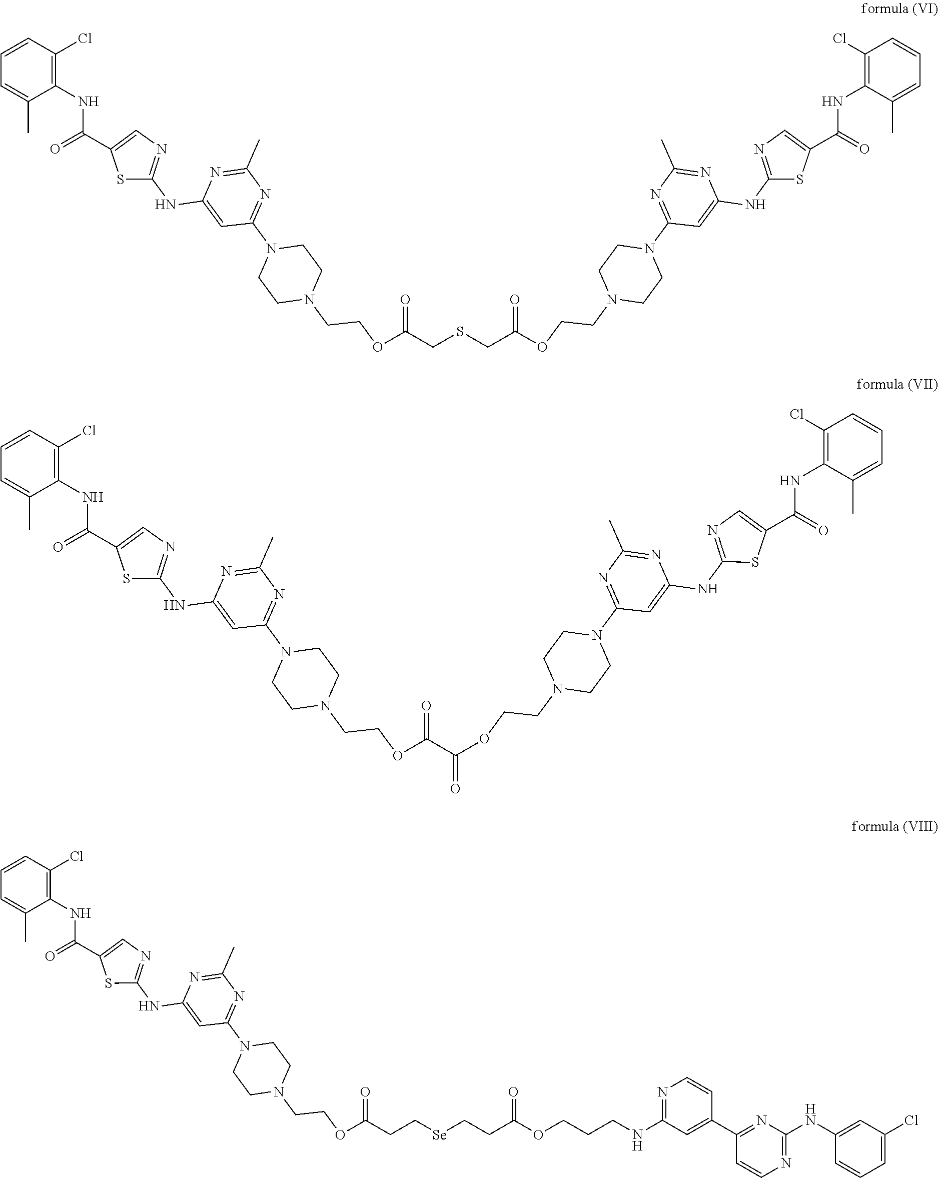 [SMILES: Cc1nc(Nc2ncc(C(=O)Nc3c(C)cccc3Cl)s2)cc(N2CCN(CCOC(=O)CSCC(=O)OCCN3CCN(c4cc(Nc5ncc(C(=O)Nc6c(C)cccc6Cl)s5)nc(C)n4)CC3)CC2)n1 ; Cc1nc(Nc2ncc(C(=O)Nc3c(C)cccc3Cl)s2)cc(N2CCN(CCOC(=O)C(=O)OCCN3CCN(c4cc(Nc5ncc(C(=O)Nc6c(C)cccc6Cl)s5)nc(C)n4)CC3)CC2)n1 ; Cc1nc(Nc2ncc(C(=O)Nc3c(C)cccc3Cl)s2)cc(N2CCN(CCOC(=O)CC[Se]CCC(=O)OCCCNc3cc(-c4ccnc(Nc5cccc(Cl)c5)n4)ccn3)CC2)n1]

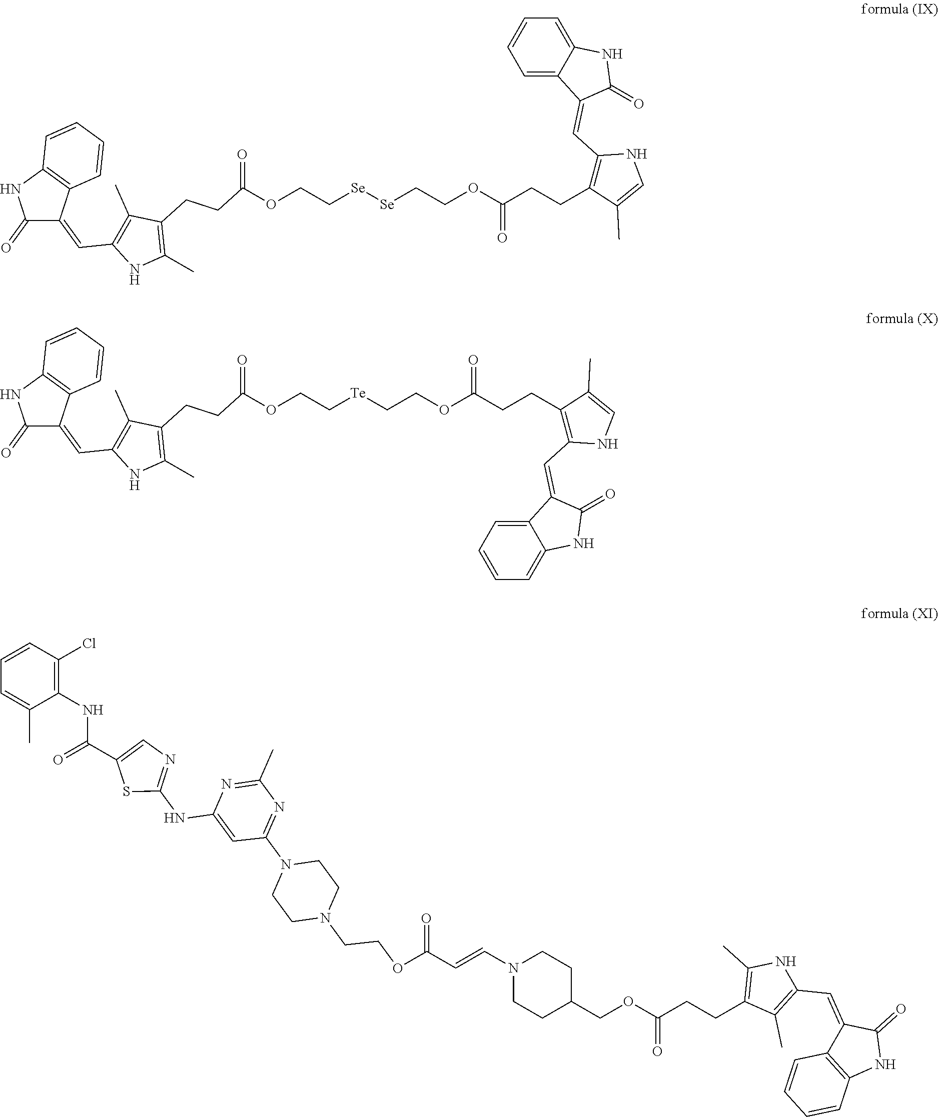

In certain embodiments, a composition comprising a singlet oxygen-cleavable anti-VEGF prodrug, a photosensitizer, and a biocompatible polymer can be synthesized. The dimeric prodrug with enhanced hydrophobicity compared with its monomeric molecule can be co-assembled with photosensitizer, such as, for example, FDA-approved verteporfin, temoporfin, padoporfin, 2-[1-hexyloxyethyl]-2-devinyl pyropheophorbide-a (HPPH), chlorin e6 and analogs thereof, including, for example, talaporfin, diaspartyl chlorin e6, or monoseryl chlorin e6, and biocompatible polymer, such as, for example, DSPE-PEG, to form multifunctional nanoparticles. In certain embodiments, the multifunctional particle is Di-DAS-VER, which is the singlet oxygen-cleavable anti-VEGF prodrug of two molecules of dasatinib linked with a thioketal linker in a DSPE-PEG₂₀₀₀-based nanoparticle with verteporfin.

In certain embodiments, dasatinib (DAS), an SRC family kinase and multi-kinase inhibitor with strong antiangiogenic efficacy, can be used for prodrug fabrication. The dimeric prodrug (Di-DAS) can be synthesized through condensation reactions with thioketal-containing dipropionic acid. The synthesis route of the prodrug (Di-DAS) is provided in FIG. 7.

In certain embodiments, the prodrug-based nanoparticles can be prepared by the nanoprecipitation method, with a photosensitizer, such as, for example, verteporfin and polymers at optimized feeding ratios of 1/15verteporfin: Di-DAS (wt/wt) and about 1/5to about 1/2, preferably about 1/5, polymer: Di-DAS (wt/wt). The particle size of the prepared nanoparticles can be about 50 nm to about 200 nm, about 50 nm to about 150 nm, about 68.3 nm to about 131.3 nm, or about 131.3 nm, with a relatively low polydispersity index (PDI) of about 0.01 to about 0.30, about 0.10 to about 0.30, about 0.138 to about 0.295, or about 0.15. In certain embodiments, the loading capacity of the nanoparticles can be about 20% to about 80%, about 40% to about 70%, about 41.97% to about 69.82%, or about 69.82% for Di-DAS and about 0.50% to about 20%, about 1% to about 3.5%, about 1.23% to about 3.26%, or about 3.26% for verteporfin.

In certain embodiments, the photosensitizer can be activated by light, such as, for example, red or near infrared light (about 600 nm to about 900 nm, about 620 nm to about 760 nm, or preferably about 690 nm for verteporfin, about 652 nm for temoporfin, about 655 nm for HPPH, about 660 nm for chlorin e6 and analogs thereof, or about 760 nm for padoporfin). In certain embodiments, the light can be applied to a subject after administration of the compound of the subject invention.

In one embodiment, the subject compositions are formulated as an orally-consumable product, such as, for example a food item, capsule, pill, or drinkable liquid. An orally deliverable pharmaceutical is any physiologically active substance delivered via initial absorption in the gastrointestinal tract or into the mucus membranes of the mouth. The topic compositions can also be formulated as a solution that can be administered via, for example, injection, which includes intravenously, intraperitoneally, intramuscularly, intrathecally, or subcutaneously. In other embodiments, the subject compositions are formulated to be administered via the skin through a patch or directly onto the skin for local or systemic effects. The compositions can be administered sublingually, buccally, rectally, or vaginally. Furthermore, the compositions can be sprayed into the nose for absorption through the nasal membrane, nebulized, inhaled via the mouth or nose, or administered in the eye or ear.

Orally consumable products according to the invention are any preparations or compositions suitable for consumption, for nutrition, for oral hygiene, or for pleasure, and are products intended to be introduced into the human or animal oral cavity, to remain there for a certain period of time, and then either be swallowed (e.g., food ready for consumption or pills) or to be removed from the oral cavity again (e.g., chewing gums or products of oral hygiene or medical mouth washes). While an orally-deliverable pharmaceutical can be formulated into an orally consumable product, and an orally consumable product can comprise an orally deliverable pharmaceutical, the two terms are not meant to be used interchangeably herein.

Orally consumable products include all substances or products intended to be ingested by humans or animals in a processed, semi-processed, or unprocessed state. This also includes substances that are added to orally consumable products (particularly food and pharmaceutical products) during their production, treatment, or processing and intended to be introduced into the human or animal oral cavity.

Orally consumable products can also include substances intended to be swallowed by humans or animals and then digested in an unmodified, prepared, or processed state; the orally consumable products according to the invention therefore also include casings, coatings, or other encapsulations that are intended to be swallowed together with the product or for which swallowing is to be anticipated.

In one embodiment, the orally consumable product is a capsule, pill, syrup, emulsion, or liquid suspension containing a desired orally deliverable substance. In one embodiment, the orally consumable product can comprise an orally deliverable substance in powder form, which can be mixed with water or another liquid to produce a drinkable orally-consumable product.

In some embodiments, the orally-consumable product according to the invention can comprise one or more formulations intended for nutrition or pleasure. These particularly include baking products (e.g., bread, dry biscuits, cake, and other pastries), sweets (e.g., chocolates, chocolate bar products, other bar products, fruit gum, coated tablets, hard caramels, toffees and caramels, and chewing gum), alcoholic or non-alcoholic beverages (e.g., cocoa, coffee, green tea, black tea, black or green tea beverages enriched with extracts of green or black tea, Rooibos tea, other herbal teas, fruit-containing lemonades, isotonic beverages, soft drinks, nectars, fruit and vegetable juices, and fruit or vegetable juice preparations), instant beverages (e.g., instant cocoa beverages, instant tea beverages, and instant coffee beverages), meat products (e.g., ham, fresh or raw sausage preparations, and seasoned or marinated fresh meat or salted meat products), eggs or egg products (e.g., dried whole egg, egg white, and egg yolk), cereal products (e.g., breakfast cereals, muesli bars, and pre-cooked instant rice products), dairy products (e.g., whole fat or fat reduced or fat-free milk beverages, rice pudding, yoghurt, kefir, cream cheese, soft cheese, hard cheese, dried milk powder, whey, butter, buttermilk, and partly or wholly hydrolyzed products containing milk proteins), products from soy protein or other soy bean fractions (e.g., soy milk and products prepared thereof, beverages containing isolated or enzymatically treated soy protein, soy flour containing beverages, preparations containing soy lecithin, fermented products such as tofu or tempeh products prepared thereof and mixtures with fruit preparations and, optionally, flavoring substances), fruit preparations (e.g., jams, fruit ice cream, fruit sauces, and fruit fillings), vegetable preparations (e.g., ketchup, sauces, dried vegetables, deep-freeze vegetables, pre-cooked vegetables, and boiled vegetables), snack articles (e.g., baked or fried potato chips (crisps) or potato dough products and extrudates on the basis of maize or peanuts), products on the basis of fat and oil or emulsions thereof (e.g., mayonnaise, remoulade, and dressings), other ready-made meals and soups (e.g., dry soups, instant soups, and pre-cooked soups), seasonings (e.g., sprinkle-on seasonings), sweetener compositions (e.g., tablets, sachets, and other preparations for sweetening or whitening beverages or other food). The present compositions may also serve as semi-finished products for the production of other compositions intended for nutrition or pleasure.

The subject composition can further comprise one or more pharmaceutically acceptable carriers, and/or excipients, and can be formulated into preparations, for example, solid, semi-solid, liquid, or gaseous forms, such as tablets, capsules, powders, granules, ointments, solutions, suppositories, injections, inhalants, and aerosols.

The term “pharmaceutically acceptable” as used herein means compatible with the other ingredients of a pharmaceutical composition and not deleterious to the recipient thereof.

Carriers and/or excipients according the subject invention can include any and all solvents, diluents, buffers (such as, e.g., neutral buffered saline, phosphate buffered saline, or optionally Tris-HCl, acetate or phosphate buffers), oil-in-water or water-in-oil emulsions, aqueous compositions with or without inclusion of organic co-solvents suitable for, e.g., IV use, solubilizers (e.g., Polysorbate 65, Polysorbate 80), colloids, dispersion media, vehicles, fillers, chelating agents (e.g., EDTA or glutathione), amino acids (e.g., glycine), proteins, disintegrants, binders, lubricants, wetting agents, emulsifiers, sweeteners, colorants, flavorings, aromatizers, thickeners (e.g. carbomer, gelatin, or sodium alginate), coatings, preservatives (e.g., Thimerosal, benzyl alcohol, polyquaterium), antioxidants (e.g., ascorbic acid, sodium metabisulfite), tonicity controlling agents, absorption delaying agents, adjuvants, bulking agents (e.g., lactose, mannitol) and the like. The use of carriers and/or excipients in the field of drugs and supplements is well known. Except for any conventional media or agent that is incompatible with the target health-promoting substance or with the composition, carrier or excipient use in the subject compositions may be contemplated.

In one embodiment, the compositions of the subject invention can be made into aerosol formulations SO that, for example, it can be nebulized or inhaled. Suitable pharmaceutical formulations for administration in the form of aerosols or sprays are, for example, powders, particles, solutions, suspensions or emulsions. Formulations for oral or nasal aerosol or inhalation administration may also be formulated with carriers, including, for example, saline, polyethylene glycol or glycols, DPPC, methylcellulose, or in mixture with powdered dispersing agents or fluorocarbons. Aerosol formulations can be placed into pressurized propellants, such as dichlorodifluoromethane, propane, nitrogen, fluorocarbons, and/or other solubilizing or dispersing agents known in the art. Illustratively, delivery may be by use of a single-use delivery device, a mist nebulizer, a breath-activated powder inhaler, an aerosol metered-dose inhaler (MDI), or any other of the numerous nebulizer delivery devices available in the art. Additionally, mist tents or direct administration through endotracheal tubes may also be used.

In one embodiment, the compositions of the subject invention can be formulated for administration via injection, for example, as a solution or suspension. The solution or suspension can comprise suitable non-toxic, parenterally-acceptable diluents or solvents, such as mannitol, 1,3-butanediol, water, Ringer's solution, or isotonic sodium chloride solution, or suitable dispersing or wetting and suspending agents, such as sterile, non-irritant, fixed oils, including synthetic mono- or diglycerides, and fatty acids, including oleic acid. One illustrative example of a carrier for intravenous use includes a mixture of 10% USP ethanol, 40% USP propylene glycol or polyethylene glycol 600 and the balance USP Water for Injection (WFI). Other illustrative carriers for intravenous use include 10% USP ethanol and USP WFI; 0.01-0.1% triethanolamine in USP WFI; or 0.01-0.2% dipalmitoyl diphosphatidylcholine in USP WFI; and 1-10% squalene or parenteral vegetable oil-in-water emulsion. Water or saline solutions and aqueous dextrose and glycerol solutions may be preferably employed as carriers, particularly for injectable solutions. Illustrative examples of carriers for subcutaneous or intramuscular use include phosphate buffered saline (PBS) solution, 5% dextrose in WFI and 0.01-0.1% triethanolamine in 5% dextrose or 0.9% sodium chloride in USP WFI, or a 1 to 2 or 1 to 4 mixture of 10% USP ethanol, 40% propylene glycol and the balance an acceptable isotonic solution such as 5% dextrose or 0.9% sodium chloride; or 0.01-0.2% dipalmitoyl diphosphatidylcholine in USP WFI and 1 to 10% squalene or parenteral vegetable oil-in-water emulsions.

In one embodiment, the compositions of the subject invention can be formulated for administration via topical application onto the skin, for example, as topical compositions, which include rinse, spray, or drop, lotion, gel, ointment, cream, foam, powder, solid, sponge, tape, vapor, paste, tincture, or using a transdermal patch. Suitable formulations of topical applications can comprise in addition to any of the pharmaceutically active carriers, for example, emollients such as carnauba wax, cetyl alcohol, cetyl ester wax, emulsifying wax, hydrous lanolin, lanolin, lanolin alcohols, microcrystalline wax, paraffin, petrolatum, polyethylene glycol, stearic acid, stearyl alcohol, white beeswax, or yellow beeswax. Additionally, the compositions may contain humectants such as glycerin, propylene glycol, polyethylene glycol, sorbitol solution, and 1,2,6 hexanetriol or permeation enhancers such as ethanol, isopropyl alcohol, or oleic acid.

### Methods of Using Compounds of the Subject Invention

In certain embodiments, a prodrug or nanoparticle composition, such as, for example, Di-DAS or derivatives thereof or Di-DAS-VER can be administered to a subject. Any means of administration that can permit Di-DAS or derivatives thereof or Di-DAS-VER to contact cells in vitro or in vivo, including, for example, orally, intravenously, intraperitoneally, intramuscularly, intrathecally, or subcutaneously.

In certain embodiments, after administration, Di-DAS or derivatives thereof or Di-DAS-VER can release DAS upon light irradiation of the subject. Further the photosensitizer of the nanoparticle can be activated by light irradiation. In certain embodiments, the activation of the photosensitizer can release singlet oxygen, which release DAS. In certain embodiments, the wavelength of the light can be about 100 nm to about 1000 nm, about 500 nm to about 900 nm, about 620 nm to about 750 nm, or, preferably, about 690 nm. In certain embodiments, the subject, nanoparticle, and/or prodrug can be irradiated for about 1 s to about 24 hours, about 10 s to about 12 hours, about 15 s to about 1 hour, about 30 s to about 30 min, about 45 s to about 10 min, about 1 min to about 5 min, or about 1 min. In certain embodiments, an entire subject can be irradiated. Alternatively, a specific portion of a subject can be irradiated, such as, for example, eye, head, face, leg, arm, wrist, chest, abdomen, neck, or calf. In certain embodiments, the subject can be irradiated before, during, or after administration of the compounds or compositions of the subject invention. In certain embodiments, the irradiation of the prodrug, nanoparticle and/or subject can occur at least 1 s, 2 s, 5 s, 10 s, 15 s, 30 s, 45 s, 1 min, 2 min, 5 min, 10 min, 15 min, 30 min, 45 min, 1 hour, 2 hours, 5 hours, or 10 hours after administration of the compounds or compositions of the subject invention. In certain embodiments, the subject, nanoparticle, and/or prodrug can be irradiated after the prodrug is at a specific location, such as, for example, tumor, organ, including an eye, or tissue. In certain embodiments, the irradiance can be about 1 mW/cm² to about 1000 mW/cm², 5 mW/cm² to about 500 mW/cm², 10 mW/cm² to about 250 mW/cm², 15 mW/cm² to about 150 mW/cm², 25 mW/cm² to about 125 mW/cm², or about 100 mW/cm². The light can be delivered by any natural or artificial source capable of providing light at the wavelength and irradiance for the described amount of time, including, for example, laser, incandescent, halogen, fluorescent, or light emitting diode (LED).

In preferred embodiments, the subject compound or compositions can be irradiated to photoactivate the photosensitizer, such as, for example, verteporfin. In certain embodiments, the irradiation of the photosensitizer can generate singlet oxygen for the cleavage of dimeric prodrug. The cleavage can trigger a cascaded anti-VEGF agent release, leading to combined antiangiogenic therapy against CNV. The photoactivation of the photosensitizer can also lead to photodynamic therapy (PDT) against CNV by damaging abnormal neovascular endothelial cells through singlet oxygen production.

In certain embodiments, the prodrug, photosensitizer, and/or nanoparticle is non-toxic before activation by light. Upon light irradiation of the prodrug, photosensitizer, and/or nanoparticle, preferably at 690 nm, an anti-VEGF agent can be released from the prodrug or composition containing the photosensitizer and prodrug, which can inhibit VEGF. Additionally, the light irradiation can activate the photosensitizer, which can cause the photosensitizer to become toxic to specific tissues and/or cell. In certain embodiments, temporally and spatially selective VEGF inhibition and photosensitizer activation could be achieved by activating prodrug, photosensitizer, and/or nanoparticle molecules with light.

In certain embodiments, prodrugs can realize site specific release of an anti-VEGF agent and/or photosensitizer, such as, for example, DAS or VER. In certain embodiments, the location and/or method of administration of the prodrug and photosensitizer or composition thereof can be flexibly adjusted according to the target disease, which can minimize side effects. In certain embodiments, the prodrug and photosensitizer are not cytotoxic; the minimal toxicity of the prodrug and photosensitizer or composition thereof permits a broad therapeutic window of anti-VEGF agents and photosensitizers. In certain embodiments of the subject invention, the activity of anti-VEGF agents and photosensitizers can be externally modulated with light. In certain embodiments, the red light-triggered activation (620 nm to 750 nm) of the prodrug can be achieved by utilizing singlet oxygen-sensitive cleavage of thioketal linkage, which exposes retinas to less harmful light compared with short-wavelength light.

In certain embodiments, the subject compound or compositions, specifically the Di-DAS-VER nanoparticle can inhibit endothelial cells tube growth upon light irradiation. In certain embodiments, the subject compound or compositions, specifically the Di-DAS-VER nanoparticles can exhibit significantly enhanced cytotoxicity when exposed to light irradiation, demonstrating the superior anti-angiogenic efficacy of multifunctional nanosystems.

All patents, patent applications, provisional applications, and publications referred to or cited herein are incorporated by reference in their entirety, including all figures and tables, to the extent they are not inconsistent with the explicit teachings of this specification.

Following are examples that illustrate procedures for practicing the invention. These examples should not be construed as limiting. All percentages are by weight and all solvent mixture proportions are by volume unless otherwise noted.

### EXAMPLE 1

### Development of Prodrug-Based Nanosystem

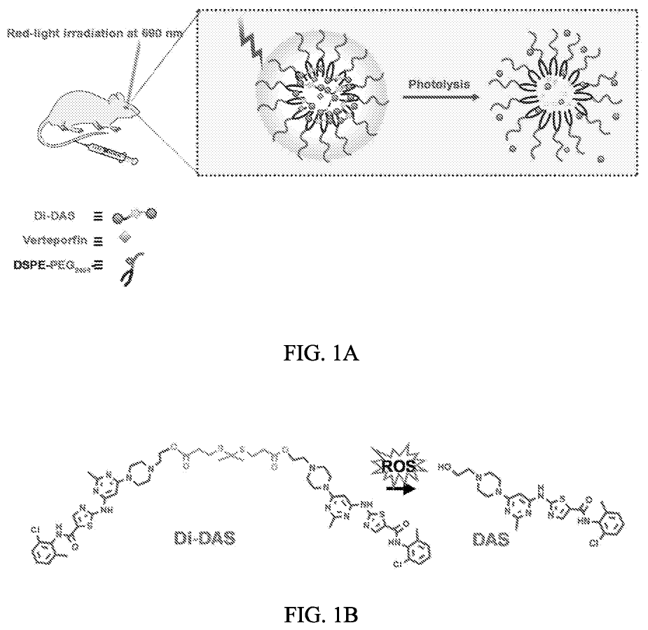

A noninvasive photoactivatable prodrug-based nanosystem was developed for combined anti-angiogenic and anti-vascular treatment targeting wet AMD (FIGS. 1A-1B), which enables site-specific and controllable activation of anti-VEGF prodrugs upon light irradiation to the eye. A reactive oxygen species (ROS)-cleavable dimeric prodrug was synthesized by conjugating two molecules of anti-VEGF agent via a thioketal linker. The dimeric prodrug with enhanced hydrophobicity compared with its monomeric molecule can be easily co-assembled with FDA-approved photosensitizer (verteporfin) and biocompatible polymer (DSPE-PEG₂₀₀₀) to form multifunctional nanoparticles (Di-DAS-VER). Since the verteporfin is activated by red light, this prodrug-based nanosystem is also expected to be triggered by red light, which is ideal for ocular use and thus minimizes both local and systemic side effects through noninvasive intravenous administration. When applying red-light irradiation to the eye, the photoactivated verteporfin can generate singlet oxygen for the cleavage of dimeric prodrug and trigger a cascaded anti-VEGF agent release, leading to combined antiangiogenic therapy and PDT against CNV.

### EXAMPLE 2

### Preparing Di-Das and Verteporfin Prodrug

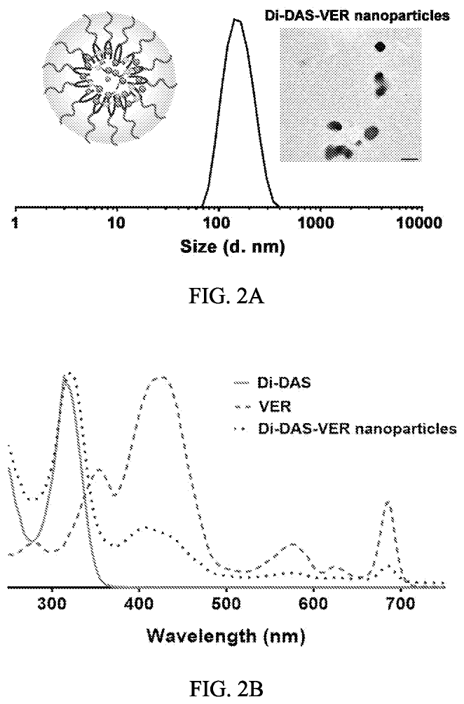

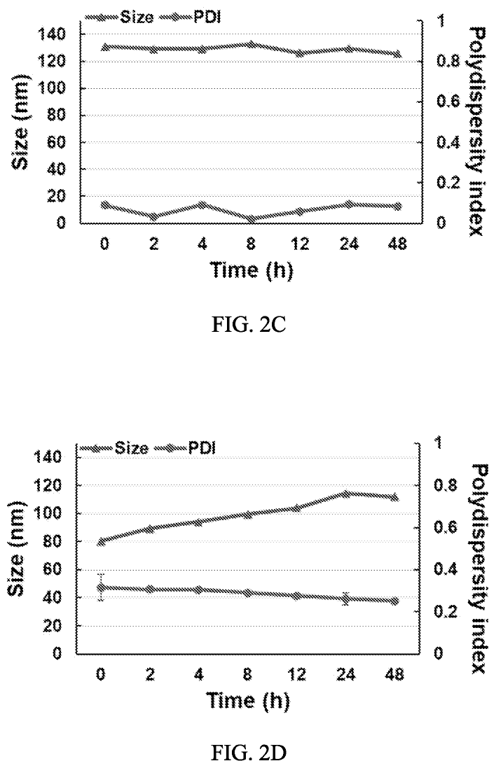

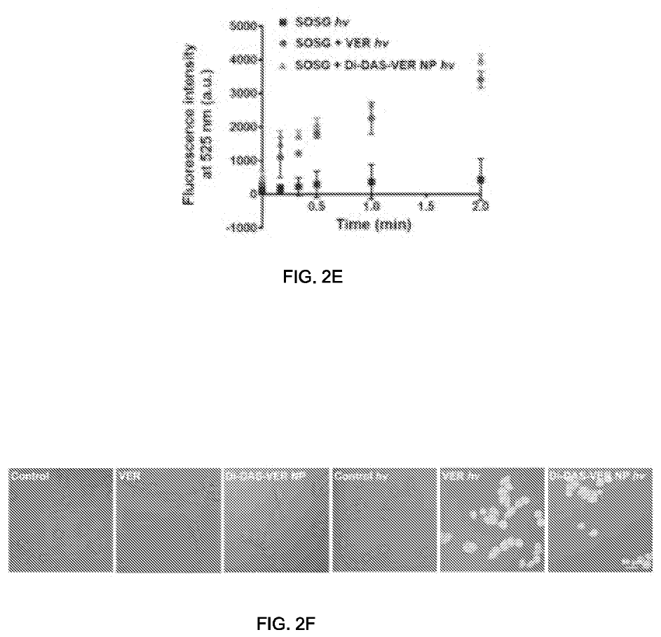

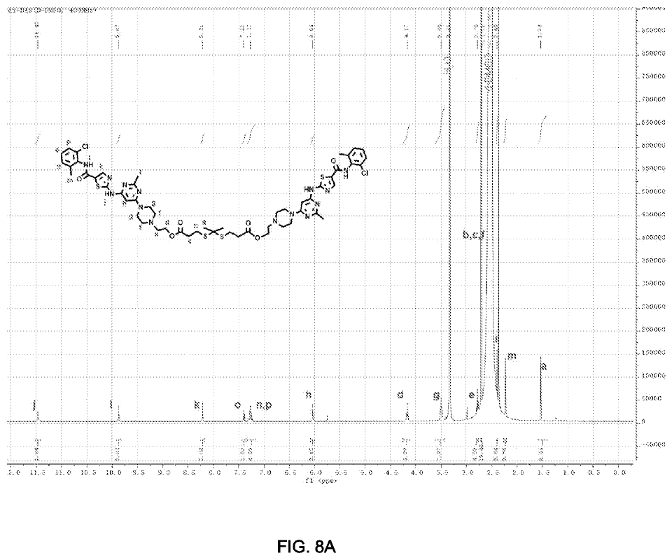

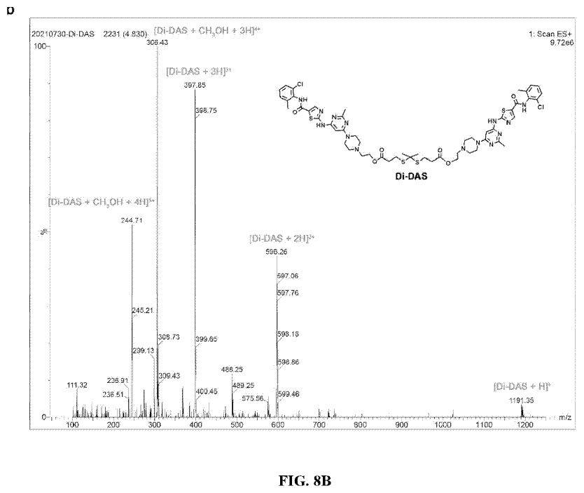

As a proof of concept, dasatinib (DAS), a SRC family kinase and multi-kinase inhibitor with strong antiangiogenic efficacy¹⁸, was chosen for prodrug fabrication. The synthesis route of the dimeric prodrug (Di-DAS) was showed in FIG. 7. The product was confirmed by nuclear magnetic resonance hydrogen (¹H NMR) spectroscopy and mass spectroscopy (FIG. 8). We subsequently prepared prodrug-based nanoparticles by the nanoprecipitation method, with verteporfin and polymers at optimized feeding ratios of 1/15 (verteporfin: Di-DAS, wt/wt) and ⅕ (polymer: Di-DAS, wt/wt) relative to the prodrug, respectively. The particle size of the prepared nanoparticles was about 131.3 nm, with a relatively low polydispersity index (PDI) of 0.15 (FIG. 2A). The spherical morphology of Di-DAS-VER nanoparticles were further confirmed by transmission electron microscopy (TEM) image (FIG. 2A). Additionally, the nanoparticles exhibited characteristic absorption peaks of Di-DAS and verteporfin at 320 nm and 690 nm (FIG. 2B), ascribing to successful co-assembly of the two molecules. The loading capacity was 69.82% for Di-DAS and 3.26% for verteporfin determined by high performance liquid chromatography (HPLC) analysis, respectively. DLS results showed the nanoparticles could be stable in PBS or DMEM containing 10% fetal bovine serum (FBS) at 37° C. for 48 h (FIGS. 2C-2D), indicating its favorable stability in biological buffer. As shown in FIG. 2E, a time-dependent increase in SOSG fluorescence intensity at 525 nm could be observed in Di-DAS-VER nanoparticles plus irradiation group, similar to that of the verteporfin plus irradiation group, indicating that ¹O₂ generation ability of Di-DAS-VER nanoparticles was comparable to that of free verteporfin. Furthermore, we also examined the in vitro ROS generation ability of the nanoparticles using 2′,7′-dichlorfluorescein diacetate (DCFH-DA) probe. Human umbilical vein endothelial cells (HUVEC), which commonly serve as in vitro endothelial cell model of CNV, were used for in vitro evaluations. As shown in FIG. 2F, little DCF fluorescence signal could be observed within HUVECs after exposure to irradiation only, or treatment with different formulations without light exposure. As a comparison, cells treated with Di-DAS-VER nanoparticles plus irradiation exhibited strong DCF fluorescence, which validated that Di-DAS-VER nanoparticles treatment had an efficient ROS generation upon red-light irradiation in vitro.

### EXAMPLE 3

### Di-Das and Verteporfin Prodrug Irradiation

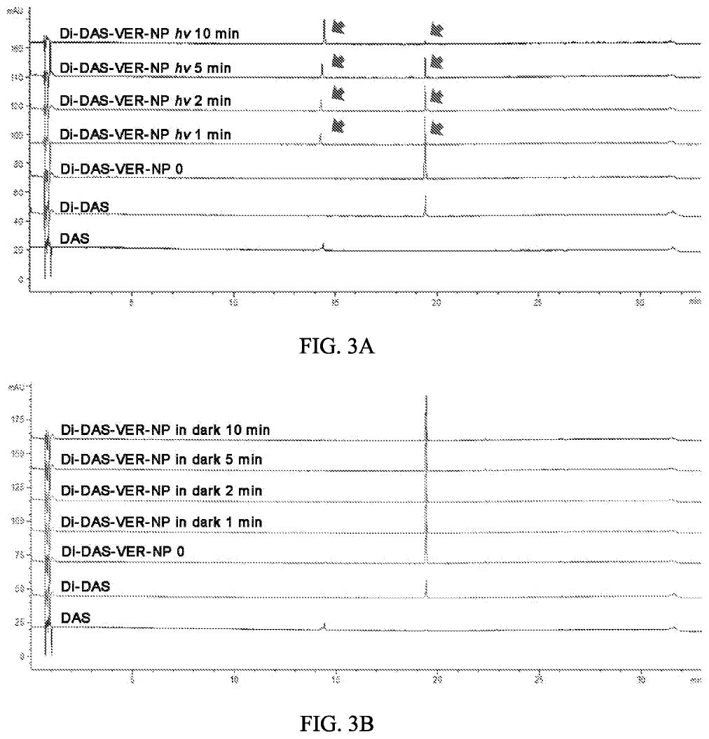

To further evaluate whether light irradiation could trigger the cleavage of the dimeric prodrugs to generate active anti-VEGF agents, Di-DAS-VER nanoparticle solutions were irradiated with a 690 nm laser (100 mW/cm²) for different time periods before HPLC detection. As shown in FIGS. 3A-3B, the peak at 19.4 min, corresponding to Di-DAS, decreased dramatically, while the new peak located at 14.2 min, corresponding to DAS, emerged and gradually increased upon exposure to red-light irradiation. In the contrast, almost no Di-DAS degraded, and no DAS generated during the same periods without illumination. The results clearly demonstrated that the prodrug-based nanosystem could be activated and generate free DAS in response to light irradiation.

### EXAMPLE 4

### Anti-Angiogenic Activity of Das and Di-Das

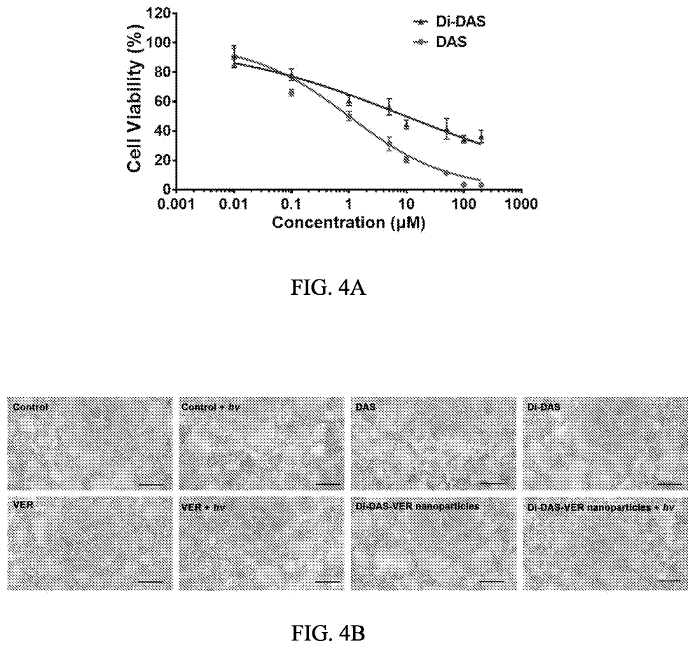

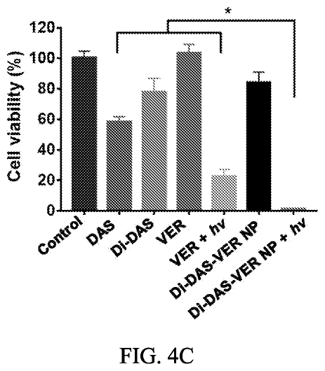

We tested the in vitro anti-angiogenic activity of DAS and Di-DAS in VEGF-stimulated human umbilical vein endothelial cells (HUVECs). As expected, Di-DAS showed attenuated anti-VEGF ability than DAS, evidenced by the result that the 50% inhibition concentration (IC₅₀) for Di-DAS was 9.86 μM, about 10-fold higher than that of DAS (0.91 μM) (FIG. 4A). To evaluate the anti-angiogenic capacity of the prodrug-based nanosystem before and after light irradiation, VEGF-induced HUVEC tube formation assay was performed when treated with different formulations at a DAS equivalent dose of 0.05 μM. As shown in FIG. 4B, Di-DAS and verteporfin could not inhibit VEGF-induced HUVEC differentiation and formation of vessel-like tubes. Notably, Di-DAS-VER nanoparticle treatment resulted in significant inhibition of tube growth upon light irradiation, comparable to that of DAS, whereas it had little impact on VEGF-stimulated HUVEC tube growth in the dark, indicating that the prodrug-based nanosystem could minimize undesired systemic anti-VEGF effects before light-triggered activation of the dimeric prodrug. To assess therapeutic efficacy of combined anti-VEGF and PDT treatment, thiazolyl blue tetrazolium bromide (MTT) assay was conducted for measuring VEGF-stimulated HUVEC cell viability. Compared with DAS treatment and verteporfin treatment followed by irradiation, Di-DAS-VER nanoparticles exhibited significantly enhanced cytotoxicity when exposed to 4 J/cm² (10 mW/cm², 400 s) of light irradiation at 690 nm (FIG. 4C), demonstrating the superior anti-angiogenic efficacy of the multifunctional nanosystems.

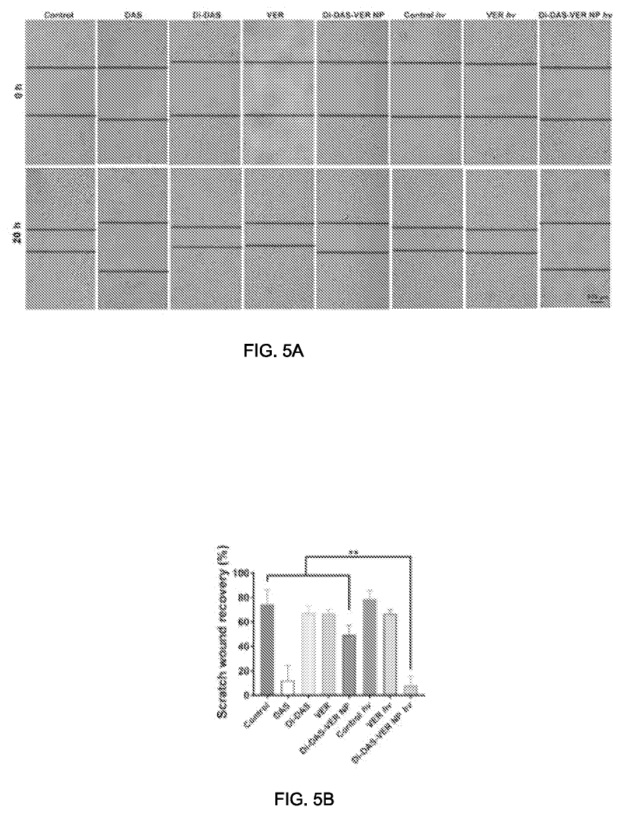

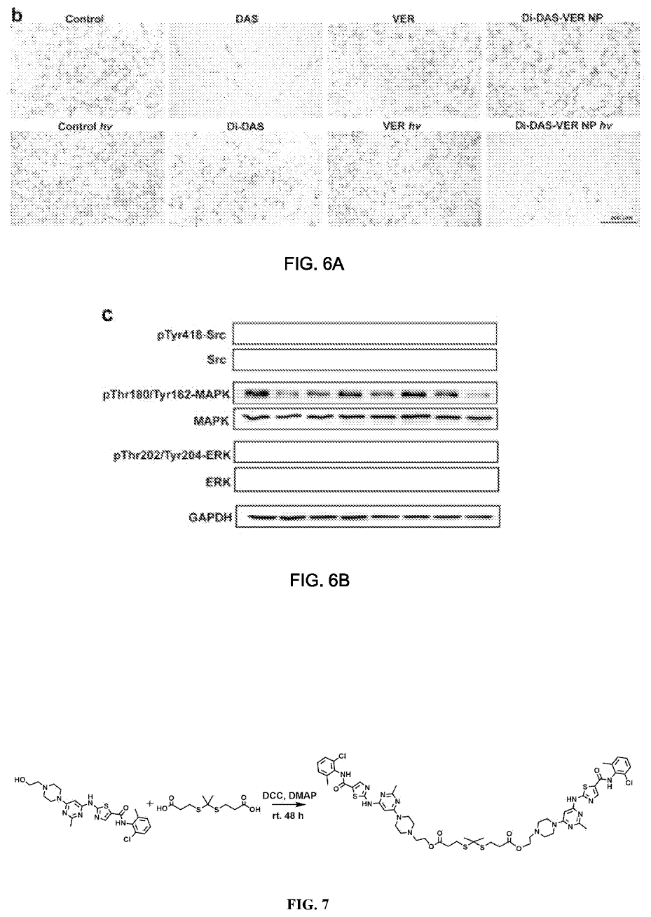

Encouraged by satisfactory results on light-triggered inhibition of endothelial tube-forming potential, we further explored the influence of Di-DAS-VER nanoparticles on the migration of VEGF-stimulated HUVECs by wound healing and transwell assays. Compared with Di-DAS and Di-DAS-VER nanoparticle treatment without light exposure that still led to 67.6% and 49.8% of scratch wound recovery, Di-DAS-VER nanoparticles plus irradiation severely impaired recovery of the generated scratches on HUVEC monolayer to 7.83%, comparable to that of DAS group (11.35%) (FIGS. 5A-B). Additionally, the cell number of HUVECs on the bottom side of the transwell, which represented the endothelial cells migrating across transwell membrane towards serum-containing medium, was slightly decreased by Di-DAS-VER nanoparticle incubation in the dark, whereas Di-DAS-VER nanoparticle plus light irradiation completely hindered HUVECs moving towards the lower compartment of transwell (FIG. 6A). Generally, all these results suggested that the anti-angiogenic ability of Di-DAS-VER nanoparticles could be efficiently recovered after red-light illumination.

Furthermore, we also checked the downstream targets of Src family kinase involved in HUVECs proliferation and survival by Western blot analysis. As presented in FIG. 6B, Di-DAS-VER nanoparticle treatments plus irradiation significantly inhibited phosphorylation of extracellular signal-regulated kinase (ERK) and mitogen-activated protein kinase (MAPK), of which activity is essential for endothelial cell survival and vessel sprouting, as compared to the untreated cells. Besides, decreased levels of phosphorylated Akt protein, a key signaling target in cell proliferation and survival, can be observed in Di-DAS-VER nanoparticles plus irradiation group, which further corroborated the photoactivatable anti-angiogenic efficacy of Di-DAS-VER nanoparticles.

The subject compositions and methods provide a novel photoactivatable nanosystem that combines anti-VEGF agents and photosensitizer for combinational treatment of wet AMD, capable of minimizing systemic toxicity and ocular adverse events while amplifying therapeutic efficacy against CNV lesions via noninvasive intravenous administration. The photoactivatable prodrugs can be easily synthesized and manipulated into nanoassemblies with high drug loading capacity and favorable stability, of which the fabrication process is simple and favors scaled-up production, clinical transformation and quality control. Furthermore, red light-triggered activation of prodrug could be achieved by utilizing singlet oxygen-sensitive cleavage of thioketal linkage, which exposes less toxicity to retinas compared with short-wavelength light.

It should be understood that the examples and embodiments described herein are for illustrative purposes only and that various modifications or changes in light thereof will be suggested to persons skilled in the art and are to be included within the spirit and purview of this application and the scope of the appended claims. In addition, any elements or limitations of any invention or embodiment thereof disclosed herein can be combined with any and/or all other elements or limitations (individually or in any combination) or any other invention or embodiment thereof disclosed herein, and all such combinations are contemplated with the scope of the invention without limitation thereto.

### REFERENCES

1. Birch, D. G.; Liang, F. Q., Age-related macular degeneration: a target for nanotechnology derived medicines. Int J Nanomedicine 2007, 2 (1), 65-77.
2. Pennington, K. L.; DeAngelis, M. M., Epidemiology of age-related macular degeneration (AMD): associations with cardiovascular disease phenotypes and lipid factors. Eye Vis(Lond) 2016, 3, 34.
3. Bora, P. S.; Hu, Z.; Tezel, T. H.; Sohn, J. H.; Kang, S. G.; Cruz, J. M.; Bora, N. S.; Garen, A.; Kaplan, H. J., Immunotherapy for choroidal neovascularization in a laser-induced mouse model simulating exudative (wet) macular degeneration. Proc Natl Acad Sci U S A 2003, 100 (5), 2679-84.
4. Ao, J.; Wood, J. P.; Chidlow, G.; Gillies, M. C.; Casson, R. J., Retinal pigment epithelium in the pathogenesis of age-related macular degeneration and photobiomodulation as a potential therapy? Clin Exp Ophthalmol 2018, 46 (6), 670-686.
5. Kulkarni, A. D.; Kuppermann, B. D., Wet age-related macular degeneration. Adv Drug Deliv Rev 2005, 57 (14), 1994-2009.
6. Tah, V.; Orlans, H. O.; Hyer, J.; Casswell, E.; Din, N.; Sri Shanmuganathan, V.; Ramskold, L.; Pasu, S., Anti-VEGF Therapy and the Retina: An Update. J Ophthalmol 2015, 2015, 627674.
7. Semeraro, F.; Morescalchi, F.; Duse, S.; Gambicorti, E.; Romano, M. R.; Costagliola, C., Systemic thromboembolic adverse events in patients treated with intravitreal anti-VEGF drugs for neovascular age-related macular degeneration: an overview. Expert Opin Drug Saf 2014, 13 (6), 785-802.
8. Choueiri, T. K.; Mayer, E. L.; Je, Y.; Rosenberg, J. E.; Nguyen, P. L.; Azzi, G. R.; Bellmunt, J.; Burstein, H. J.; Schutz, F. A., Congestive heart failure risk in patients with breast cancer treated with bevacizumab. J Clin Oncol 2011, 29 (6), 632-8.
9. Maloney, M.; Payne, S.; Herrin, J.; Sangaralingham, L.; Shah, N.; Barkmeier, A. J. O., Risk of Systemic Adverse Events after Intravitreal Bevacizumab, Ranibizumab, and Aflibercept in Routine Clinical Practice. 2020.
10. Fogli, S.; Del Re, M.; Rofi, E.; Posarelli, C.; Figus, M.; Danesi, R., Clinical pharmacology of intravitreal anti-VEGF drugs. Eye (Lond) 2018, 32 (6), 1010-1020.
11. Senra, H.; Ali, Z.; Balaskas, K.; Aslam, T. J. G. s. a. f. c.; Ophthalmologie, e. o. A. v. G. A. f. k. u. e., Psychological impact of anti-VEGF treatments for wet macular degeneration-a review. 2016, 254 (10), 1873-1880.
12. Bradley, J.; Ju, M.; Robinson, G. S., Combination therapy for the treatment of ocular neovascularization. Angiogenesis 2007, 10 (2), 141-8.
13. Tatar, O.; Adam, A.; Shinoda, K.; Stalmans, P.; Eckardt, C.; Luke, M.; Bartz-Schmidt, K. U.; Grisanti, S., Expression of VEGF and PEDF in choroidal neovascular membranes following verteporfin photodynamic therapy. Am J Ophthalmol 2006, 142 (1), 95-104.
14. Lazic, R.; Gabric, N., Verteporfin therapy and intravitreal bevacizumab combined and alone in choroidal neovascularization due to age-related macular degeneration. Ophthalmology 2007, 114 (6), 1179-85.
15. Gao, Y.; Yu, T.; Zhang, Y.; Dang, G. F., Anti-VEGF Monotherapy Versus Photodynamic Therapy and Anti-VEGF Combination Treatment for Neovascular Age-Related Macular Degeneration: A Meta-Analysis. Invest Ophth Vis Sci 2018, 59 (10), 4307-4317.
16. Zuluaga, M.; Mailhos, C.; Robinson, G.; Shima, D.; Gurny, R.; Lange, N. J. I. o.; science, v., Synergies of VEGF inhibition and photodynamic therapy in the treatment of age-related macular degeneration. 2007, 48 (4), 1767-72.
17. Teper, S.; Nowinska, A.; Pilat, J.; Wylegala, E. J. P.; therapy, p., Photodynamic therapy in VEGF inhibition non-responders-Pharmacogenetic study in age-related macular degeneration assessed with swept-source optical coherence tomography. 2016, 13, 108-113.
18. Seo, S.; Suh, W. J. M. v., Antiangiogenic effect of dasatinib in murine models of oxygen-induced retinopathy and laser-induced choroidal neovascularization. 2017, 23, 823.
19. Wang, J. L.; Xi, Y.; Liu, Y. L.; Wang, Z. H.; Zhang, Q., Combination of targeted PDT and anti-VEGF therapy for rat CNV by RGD-modified liposomal photocyanine and sorafenib. Invest Ophthalmol Vis Sci 2013, 54 (13), 7983-9.
20. Cai, W.; Chen, Q.; Shen, T.; Yang, Q.; Hu, W.; Zhao, P.; Yu, J., Intravenous anti-VEGF agents with RGD peptide-targeted core cross-linked star (CCS) polymers modified with indocyanine green for imaging and treatment of laser-induced choroidal neovascularization. Biomater Sci 2020, 8 (16), 4481-4491.

We claim:

1. A compound comprising two molecules of an anti-VEGF agent or two distinct anti-VEGF agents linked via a reactive oxygen species (ROS)-sensitive linker.

2. The compound of claim 1, wherein the anti-VEGF agent is dasatinib (DAS), orantinib, SU5402, and/or CGP60474.

3. The compound of claim 1, wherein the ROS-sensitive linker is a thioketal linker, thioether linker, peroxalate ester linker, aminoacrylate linker, selenide, diselenide, or a telluride-containing linker.

4. The compound of claim 1, wherein the compound is of formula (I), formula (II), formula (III), formula (IV), formula (V), formula (VI), formula (VII), formula (VIII), formula (IX), formula (X), or formula (XI):

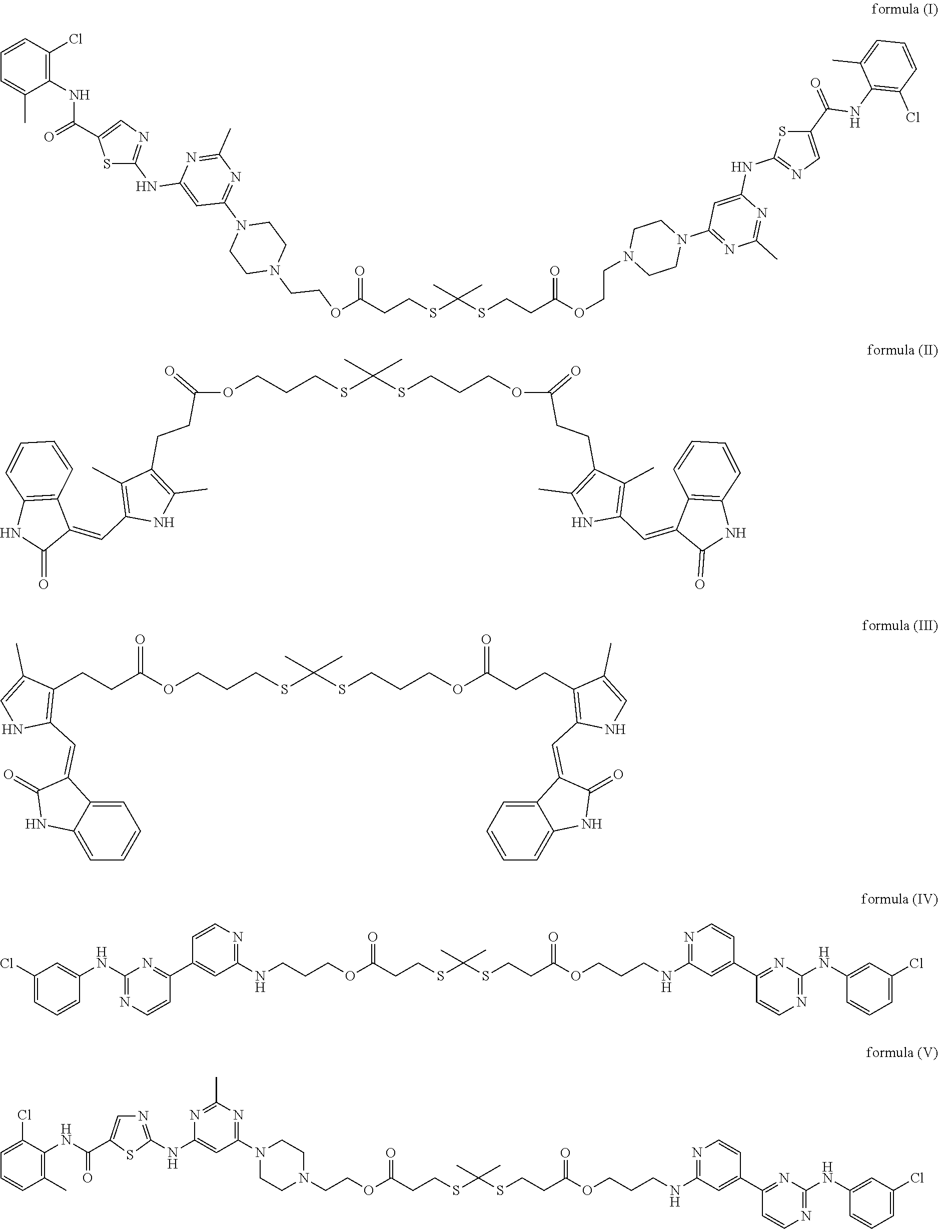

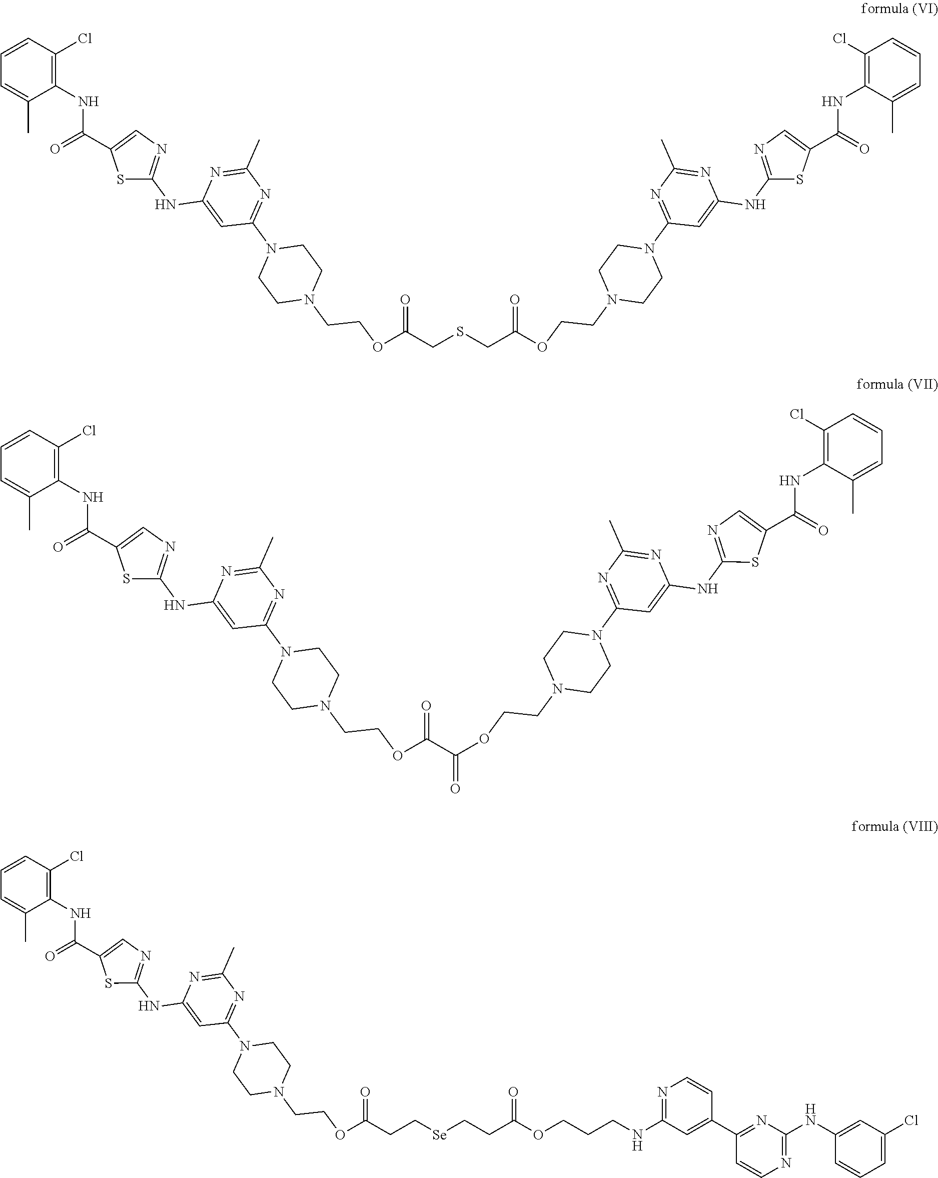 [SMILES: Cc1nc(Nc2ncc(C(=O)Nc3c(C)cccc3Cl)s2)cc(N2CCN(CCOC(=O)CSCC(=O)OCCN3CCN(c4cc(Nc5ncc(C(=O)Nc6c(C)cccc6Cl)s5)nc(C)n4)CC3)CC2)n1 ; Cc1nc(Nc2ncc(C(=O)Nc3c(C)cccc3Cl)s2)cc(N2CCN(CCOC(=O)C(=O)OCCN3CCN(c4cc(Nc5ncc(C(=O)Nc6c(C)cccc6Cl)s5)nc(C)n4)CC3)CC2)n1 ; Cc1nc(Nc2ncc(C(=O)Nc3c(C)cccc3Cl)s2)cc(N2CCN(CCOC(=O)CC[Se]CCC(=O)OCCCNc3cc(-c4ccnc(Nc5cccc(Cl)c5)n4)ccn3)CC2)n1]

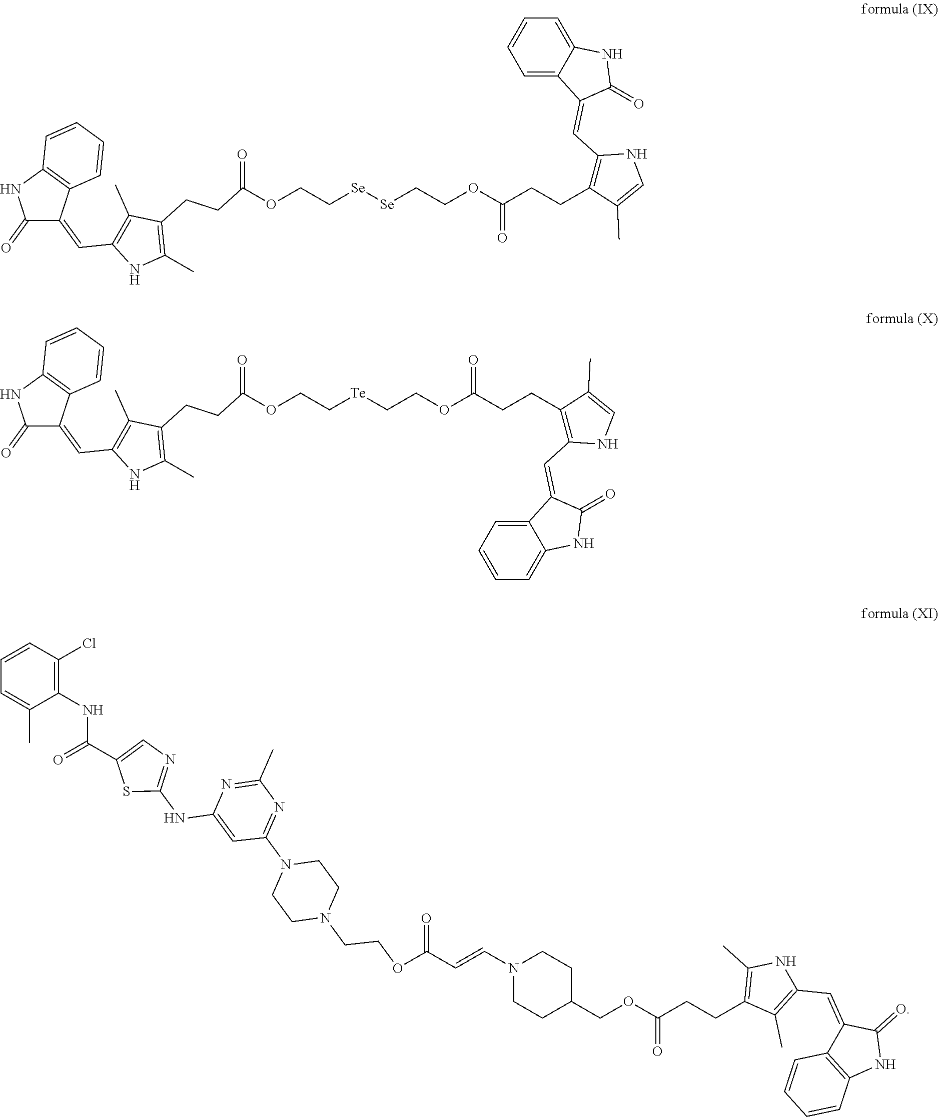

5. A composition comprising the compound of claim 1 at a concentration of about 20% to about 80% or about 41.97% to about 69.82%.

6. The composition of claim 5, further comprising a photosensitizer and/or biocompatible polymeric material.

7. The composition of claim 6, wherein the composition is a nanoparticle.

8. The composition of claim 6, wherein the photosensitizer is verteporfin, temoporfin, padoporfin, 2-[1-hexyloxyethyl]-2-devinyl pyropheophorbide-a (HPPH), chlorin e6 and analogs thereof.

9. The composition of claim 6, wherein the biocompatible polymeric material is a DSPE-PEG.

10. The composition of claim 6, wherein the composition is about 0.5% to about 20% photosensitizer.

11. The composition of claim 10, wherein the composition is about 1.23% to about 3.26% photosensitizer.

12. A method of CNV closure and anti-angiogenic therapy, said method comprising:
a) contacting the composition of claim 6 with an eye cell; and
b) irradiating said composition of claim 6, wherein the irradiation activates the photosensitizer and releases singlet oxygen from said photosensitizer and the singlet oxygen cleaves ROS-sensitive linkage.

13. The method of claim 12, further comprising:
c) inhibiting vascular endothelial growth.

14. The method of claim 12, wherein the irradiation is non-ionizing radiation at a wavelength of about 100 nm to about 1000 nm, about 500 nm to about 900 nm, about 620 nm to about 750 nm, or about 690 nm.

15. The method of claim 12, wherein the irradiation occurs for about 1 second to about 10 minutes.

16. The method of claim 12, wherein the irradiance of the irradiation is about 1 mW/cm² to about 1000 mW/cm², 5 mW/cm² to about 500 mW/cm², 10 mW/cm² to about 250 mW/cm², 15 mW/cm² to about 150 mW/cm², 25 mW/cm² to about 125 mW/cm², or about 100 mW/cm².

17. A method of inhibiting the tumor growth, said method comprising:
a) contacting the composition of claim 6 with a tumor cell; and
b) irradiating the composition of claim 6, wherein the irradiation activates the photosensitizer and releases singlet oxygen from said photosensitizer and the singlet oxygen cleaves ROS-sensitive linkage.

18. The method of claim 17, further comprising:
c) inhibiting vascular endothelial growth and tumor growth.

19. The method of claim 17, wherein the irradiation is non-ionizing radiation at a wavelength of about 100 nm to about 1000 nm, about 500 nm to about 900 nm, about 620 nm to about 750 nm, or about 690 nm.

20. The method of claim 17, wherein the irradiation occurs for about 1 second to about 10 minutes.

21. The method of claim 17, wherein the irradiance of the irradiation is about 1 mW/cm² to about 1000 mW/cm², 5 mW/cm² to about 500 mW/cm², 10 mW/cm² to about 250 mW/cm², 15 mW/cm² to about 150 mW/cm², 25 mW/cm² to about 125 mW/cm², or about 100 mW/cm².
